(19) United States
(12) Patent Application Publication
(10) Pub. No.: US 2005/0256177 A1
(43) Pub. Date: Nov. 17, 2005

(54) Phenyl derivatives for the treatment of abnormal cell growth

(75) Inventors: Matthew A. Marx, Waterford, CT (US); Chandra A. Prakash, Gales Ferry, CT (US); Zhuang Miao, East Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/131,020

(22) Filed: May 16, 2005

Related U.S. Application Data
(60) Provisional application No. 60/571,779, filed on May 17, 2004.

(51) Int. Cl. A61K 31/425; C07D 417/02

(52) U.S. Cl. 514/372; 548/213

(57) ABSTRACT

The invention relates to compounds of the formula 1 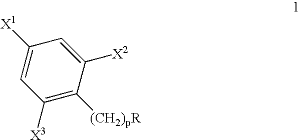
wherein X¹, X² and X³ independently is a halogen; p is an integer from 1 to 3; and R is —COOH, OR¹ wherein R¹ is a substituted isothiazole ring, or SR² wherein R² is (CH₂)_{q}C(COOH)(NHC(O)R³) wherein q is an integer from 0 to 3, and R³ is (C₁-C₄)alkyl; and to pharmaceutically acceptable salts, prodrugs and solvates thereof. The invention also relates to methods of treating a hyperproliferative disorder in a mammal by administering the compounds of formula 1 and to pharmaceutical compositions containing the compounds of formula 1.

This application claims priority to U.S. Provisional Application No. 60/571,779 filed May 17, 2004, the disclosure of which is incorporated into this application in its entirety.

### BACKGROUND OF THE INVENTION

This invention relates to novel phenyl derivatives that are useful in the treatment of hyperproliferative diseases, such as cancers, in mammals. In one embodiment, these derivatives are metabolites of 3-(4-bromo-2,6-difluoro-benzyloxy)-5-[3-(4-pyrrolidin-1-yl-butyl)-ureido]-isothiazole-4-carboxylic acid amide. This invention also relates to a method of using such compounds in the treatment of hyperproliferative diseases in mammals, especially humans, and to pharmaceutical compositions containing such compounds.

It is known that a cell may become cancerous by virtue of the transformation of a portion of its DNA into an oncogene (i.e. a gene that upon activation leads to the formation of malignant tumor cells). Many oncogenes encode proteins which are aberrant tyrosine kinases capable of causing cell transformation. Alternatively, the overexpression of a normal proto-oncogenic tyrosine kinase may also result in proliferative disorders, sometimes resulting in a malignant phenotype. It has been shown that certain tyrosine kinases may be mutated or overexpressed in many human cancers such as brain, lung, squamous cell, bladder, gastric, breast, head and neck, oesophageal, gynecological and thyroid cancers. Furthermore, the overexpression of a ligand for a tyrosine kinase receptor may result in an increase in the activation state of the receptor, resulting in proliferation of the tumor cells or endothelial cells. Thus, it is believed that inhibitors of receptor tyrosine kinases, such as the compounds of the present invention, are useful as selective inhibitors of the growth of mammalian cancer cells.

It is known that polypeptide growth factors, such as vascular endothelial growth factor (VEGF) having a high affinity to the human kinase insert-domain-containing receptor (KDR) or the murine fetal liver kinase 1 (FLK-1) receptor, have been associated with the proliferation of endothelial cells and more particularly vasculogenesis and angiogenesis. See PCT international application publication number WO 95/21613 (published Aug. 17, 1995). Agents, such as the compounds of the present invention, that are capable of binding to or modulating the KDR/FLK-1 receptor may be used to treat disorders related to vasculogenesis or angiogenesis such as diabetes, diabetic retinopathy, hemangioma, glioma, melanoma, Kaposi's sarcoma and ovarian, breast, lung, pancreatic, prostate, colon and epidermoid cancer.

Isothiazole derivatives useful as herbicides are referred to in U.S. Pat. Nos. 4,059,433 and 4,057,416, both assigned to FMC Corporation Isothiazole derivates useful in the treatment of cancer have also been disclosed in U.S. Pat. Nos. 6,235,764 and 6,548,526, and in International Patent Publications WO 99/62890 (published Dec. 9, 1999), WO 02/44158 (published Jun. 6, 2002), and WO 04/017964 (published Mar. 4, 2004).

### SUMMARY OF THE INVENTION

The present invention relates to a substantially pure compound of the formula 1 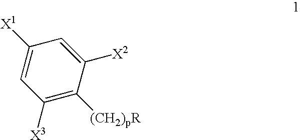
wherein:
X¹, X² and X³ independently is a halogen; p is an integer from 1 to 3; and R is OR¹ wherein R¹ is a substituted isothiazole ring or SR² wherein R² is (CH₂)_{q}C(COOH)(NHC(O)R³) wherein q is an integer from 0 to 3, and R³ is (C₁-C₄)alkyl; or a pharmaceutically acceptable salt, solvate or prodrug thereof.

In one embodiment of the invention, the compound of formula 1 is represented by formula 2 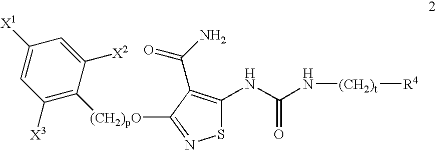
wherein t is an integer from 3 to 5; and R⁴ is —COOH, —NH(CH₂)ₖOH wherein k is an integer from 1 to 5, —NH(CH₂)ₙCOOH wherein l is an integer from 1 to 5, or a 5-membered nitrogen containing heterocycle, which may optionally be substituted by one or more oxo (═O) moieties, hydroxyl groups, or wherein an oxygen atom is bonded to the nitrogen atom of the 5-membered nitrogen containing heterocycle to form an N-oxide (N—O) group.

Other embodiments of the invention include those compounds of formula 1, wherein R is SR², q is 1, and R³ is methyl.

Other embodiments of the invention include those compounds of formula 1, wherein X¹ is bromine, and X² and X³ are fluorine.

Other embodiments of the invention include those compounds of formula 1, wherein X¹ is bromine, X² and X³ are fluorine and p is 1.

Other embodiments of the invention include those compounds of formula 2, wherein X¹ is bromine, X² and X³ are fluorine and p is 1.

Other embodiments of the invention include those compounds of formula 2, wherein X¹ is bromine, X² and X³ are fluorine, p is 1, and t is 3.

Other embodiments of the invention include those compounds of formula 2, wherein X¹ is bromine, X² and X³ are fluorine, p is 1, and t is 3, and R⁴ is —COOH.

Other embodiments of the invention include those compounds of formula 2, wherein X¹ is bromine, X² and X³ are fluorine, p is 1, and t is 4.

Other embodiments of the invention include those compounds of formula 2, wherein X¹ is bromine, X² and X³ are fluorine, p is 1, t is 4, and R⁴ is —NH(CH₂)ₖOH. Preferably k is 4.

Other embodiments of the invention include those compounds of formula 2, wherein X¹ is bromine, X² and X³ are fluorine, p is 1, t is 4, and R⁴ is —NH(CH₂)ₗCOOH. Preferably l is 3.

Other embodiments of the invention include those compounds of formula 2, wherein X¹ is bromine, X² and X³ are fluorine, p is 1, t is 4 and R⁴ is a 5-membered nitrogen containing heterocycle.

Other embodiments of the invention include those compounds of formula 2, wherein X¹ is bromine, X² and X³ are fluorine, p is 1, t is 4 and R⁴ is a 5-membered nitrogen containing heterocycle that is represented by the formula

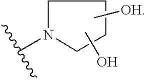

Other embodiments of the invention include those compounds of formula 2, wherein X¹ is bromine, X² and X³ are fluorine, p is 1, t is 4 and R⁴ is a 5-membered nitrogen containing heterocycle that is represented by the formula

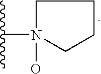

Other embodiments of the invention include those compounds of formula 2, wherein X¹ is bromine, X² and X³ are fluorine, p is 1, t is 4 and R⁴ is a 5-membered nitrogen containing heterocycle that is represented by the formula

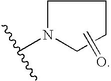

Other embodiments of the invention include those compounds of formula 2, wherein X¹ is bromine, X² and X³ are fluorine, p is 1, t is 4 and R⁴ is a 5-membered nitrogen containing heterocycle that is represented by the formula

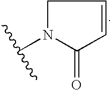

Other embodiments of the invention include those compounds of formula 1 or 2 selected from the group consisting of
4-(4-{3-[3-(4-bromo-2,6-difluoro-benzyloxy)-4-carbamoyl-isothiazol-5-yl]-ureido}-butylamino)-butyric acid;
3-(4-bromo-2,6-difluoro-benzyloxy)-5-[3-(4-pyrrolidin-1-yl-N-oxide-butyl)-ureido]-isothiazole-4-carboxylic acid amide;
2-Acetylamino 3-(4-bromo-2,6-difluoro-benzylsulfanyl)-propionic acid;
2-Acetylamino 3-(4-bromo-2,6-difluoro-benzylsulfanyl)-2-methyl-propionic acid;
3-(4-Bromo-2,6-difluoro-benzyloxy)-5-{3-[4-(4-hydroxy-butylamino)-butyl]-ureido}-isothiazole-4-carboxylic acid amide;
4-{3-[3-(4-bromo-2,6-difluoro-benzyloxy)-4-carbamoyl-isothiazol-5-yl]-ureido}-butyric acid;
3-(4-Bromo-2,6-difluoro-benzyloxy)-5-{3-[4-(2,3-dihydroxy-pyrrolidin-1-yl)-butyl]-ureido}-isothiazole-4-carboxylic acid amide;
3-(4-Bromo-2,6-difluoro-benzyloxy)-5-{3-[4-(2,4-dihydroxy-pyrrolidin-1-yl)-butyl]-ureido}-isothiazole-4-carboxylic acid amide;
3-(4-Bromo-2,6-difluoro-benzyloxy)-5-{3-[4-(3,4-dihydroxy-pyrrolidin-1-yl)-butyl]-ureido}-isothiazole-4-carboxylic acid amide;
3-(4-Bromo-2,6-difluoro-benzyloxy)-5-{3-[4-(2,5-dihydroxy-pyrrolidin-1-yl)-butyl]-ureido}-isothiazole-4-carboxylic acid amide;
3-(4-bromo-2,6-difluoro-benzyloxy)-5-{3-[4-(2-oxo-2,5-dihydro-pyrrol-1-yl)-butyl]-ureido}-isothizole-4-carboxylic acid amide;
   and the pharmaceutically acceptable salts, prodrugs, hydrates and solvates of the foregoing compounds.

The present invention also relates to a pharmaceutical composition for the treatment of a hyperproliferative disorder in a mammal which comprises a therapeutically effective amount of a compound of formula 1 or 2 and a pharmaceutically acceptable carrier.

Other embodiments of the invention include those pharmaceutical compositions wherein the hyperproliferative disorder is a cancer selected from lung cancer, bone cancer, pancreatic cancer, gastric, skin cancer, cancer of the head or neck, cutaneous or intraocular melanoma, uterine cancer, ovarian cancer, gynecological, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, squamous cell, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain, pituitary adenoma, or a combination of one or more of the foregoing cancers.

Other embodiments of the invention include those pharmaceutical compositions wherein the hyperproliferative disorder is a cancer selected from brain, lung, squamous cell, bladder, gastric, pancreatic, breast, head, neck, renal, kidney, ovarian, prostate, colorectal, oesophageal, gynecological and thyroid cancer.

Other embodiments of the invention include those pharmaceutical compositions wherein the hyperproliferative disorder is a non-cancerous hyperproliferative disorder, such as a benign hyperplasia of the skin or prostrate.

The present invention also relates to a method of treating a hyperproliferative disorder in a mammal, including a human, which comprises administering to said mammal a therapeutically effective amount of a compound of formula 1 or 2 as defined above, or a pharmaceutically acceptable salt, solvate, hydrate or prodrug thereof.

Other embodiments of the invention include those methods wherein the hyperproliferative disorder is a cancer selected from brain, squamous cell, bladder, gastric, pancreatic, breast, head, neck, oesophageal, prostate, colorectal, lung, renal, kidney, ovarian, gynecological and thyroid cancer.

Other embodiments of the invention include those methods wherein the hyperproliferative disorder is a non-cancerous hyperproliferative disorder, such as a benign hyperplasia of the skin or prostrate.

The present invention also relates to a method for the treatment of a hyperproliferative disorder in a mammal which comprises administering to said mammal a therapeutically effective amount of a compound of formula 1 or 2, as defined above, or a pharmaceutically acceptable salt, solvate, hydrate or prodrug thereof, in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, NK1 receptor antagonist, 5-HT₃ receptor antagonist, COX-2 inhibitor, an EGFR inhibitor, and anti-androgens.

The present invention also relates to a method of determining if a patient has been administered 3-(4-Bromo-2,6-difluoro-benzyloxy)-5-[3-(4-pyrrolidin-1-yl-butyl)-ureido]-isothiazole-4-carboxylic acid amide, the method comprising the step of determining if a plasma, urine, bile or fecal sample obtained from the patient shows the presence of the compound of formula 1.

The present invention also relates to kit for the treatment of a hyperproliferative disorder comprising a) a pharmaceutical composition comprising a compound of formula 1 and a pharmaceutically acceptable carrier, vehicle or diluent; and b) instructions describing a method of using the pharmaceutical composition for treating the abnormal cell growth.

Each of the patents, patent applications, published International applications, and scientific publications referred to in this patent application is incorporated herein by reference in its entirety.

### DETAILED DESCRIPTION OF THE INVENTION

The compounds of formula 1, and the pharmaceutically acceptable salts, solvates and prodrugs thereof, can also be used in combination with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined with a compound of formula 1. VEGF inhibitors are described in, for example in WO 99/62890 (published Dec. 9, 1999), U.S. Pat. No. 6,235,764 (issued May 22, 2001) and U.S. Pat. No. 6,548,526 (issued Apr. 15, 2003); WO 01/95353 (published Dec. 13, 2001), WO 02/44158 (published Jun. 6, 2002), WO 04/017964 (published Mar. 4, 2004), WO 99/24440 (published May 20, 1999), WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998). Other examples of some specific VEGF inhibitors include IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent applications: Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following U.S. provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

A compound of formula 1 may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

“Hyperproliferative disorder”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinase activation; (5) any tumors that proliferate by aberrant serine/threonine kinase activation; and (6) benign and malignant cells of other proliferative diseases in which aberrant serine/threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “alkyl”, as used herein, unless otherwise indicated, includes saturated monovalent hydrocarbon radicals having straight, cyclic (including mono- or multi-cyclic moieties) or branched moieties. It is understood that for said alkyl group to include cyclic moieties it must contain at least three carbon atoms.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

The phrase “substantially pure”, as used herein, unless otherwise indicated, refers to purity of chemical compounds wherein the compounds are preferably at least 90% pure, more preferably at least 95% pure, and most preferably at least 99% pure.

As used herein, the notation 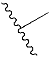
refers to a point of attachment. Thus the structure 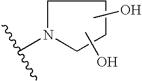
indicates that the point of attachment is the nitrogen atom.

In another embodiment of the present invention, the compounds of the claimed invention are pure and isolated.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention may have asymmetric centers and therefore may exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula 1 may also exist as tautomers. This invention relates to the use of all such tautomers and mixtures thereof.

The subject invention also includes isotopically-labelled compounds, and the pharmaceutically acceptable salts, solvates and prodrugs thereof, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The compounds of formula 1 that are basic in nature are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula 1 that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula 1. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product. Since a single compound of the present invention may include more than one acidic or basic moieties, the compounds of the present invention may include mono, di or tri-salts in a single compound.

The compounds of the present invention may be potent inhibitors of the KDRNEGF family of oncogenic and protooncogenic protein tyrosine kinase receptors, in particular VEGF, and thus are all adapted to therapeutic use as antiproliferative agents (e.g., anticancer) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that a compound of the present invention may possess activity against a range of leukemias and lymphoid malignancies.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The compounds of the present invention may also be useful as biomarkers for the metabolism of 3-(4-Bromo-2,6-difluoro-benzyloxy)-5-[3-(4-pyrrolidin-1-yl-butyl)-ureido]-isothiazole-4-carboxylic acid amide and may further be used to determine its rate of absorption and metabolic breakdown in mammals, such as humans.

The in vitro activity of the compounds of formula 1 in inhibiting the KDR/VEGF receptor may be determined by the following procedure.

The ability of the compounds of the present invention to inhibit tyrosine kinase activity may be measured using a recombinant enzyme in an assay that measures the ability of compounds to inhibit the phosphorylation of the exogenous substrate, polyGluTyr (PGT, Sigma™, 4:1). The kinase domain of the human KDRNEGF receptor (amino acids 805-1350) is expressed in Sf9 insect cells as a glutathione S-transferase (GST)-fusion protein using the baculovirus expression system. The protein is purified from the lysates of these cells using glutathione agarose affinity columns. The enzyme assay is performed in 96-well plates that are coated with the PGT substrate (0.625 μg PGT per well). Test compounds are diluted in dimethylsulfoxide (DMSO), and then added to the PGT plates so that the final concentration of DMSO in the assay is 1.6% (v/v). The recombinant enzyme is diluted in phosphorylation buffer (50 mM Hepes, pH 7.3, 125 mM NaCl, 24 mM MgCl₂). The reaction is initiated by the addition of ATP to a final concentration of 10 μM. After a 30 minute incubation at room temperature with shaking, the reaction is aspirated, and the plates are washed with wash buffer (PBS-containing 0.1% Tween-20). The amount of phosphorylated PGT is quantitated by incubation with a HRP-conjugated (HRP is horseradish peroxidase) PY-54 antibody (Transduction Labs), developed with TMB peroxidase (TMB is 3,3′,5,5′-tetramethylbenzidine), and the reaction is quantitated on a BioRad™ Microplate reader at 450 nM. Inhibition of the kinase enzymatic activity by the test compound is detected as a reduced absorbance, and the concentration of the compound that is required to inhibit the signal by 50% is reported as the IC₅₀ value for the test compound.

To measure the ability of the compounds to inhibit KDR tyrosine kinase activity for the full length protein that exists in a cellular context, the porcine aortic endothelial (PAE) cells transfected with the human KDR (Waltenberger et al., J. Biol. Chem. 269:26988, 1994) may be used. Cells are plated and allowed to attach to 96-well dishes in the same media (Ham's F12) with 10% FBS (fetal bovine serum). The cells are then washed, re-fed with serum depleted media that contains 0.1% (v/v) bovine serum albumin (BSA), and allowed to incubate for 24 hours. Immediately prior to dosing with compound, the cells are re-fed with the serum depleted media (without BSA). Test compounds, dissolved in DMSO, are diluted into the media (final DMSO concentration 0.5% (v/v)). At the end of a 2 hour incubation, VEGF₁₆₅ (50 ng/ml final) is added to the media for an 8 minute incubation. The cells are washed and lysed in HNTG buffer (20 mM Hepes, pH 7.5, 150 mM NaCl, 0.2% Triton™ X-100, 10% glycerol, 0.2 mM PMSF (phenymethylsulfonyl fluoride), 1 μg/ml pepstatin, 1 μg/ml leupeptin, 1 μg/ml aprotonin, 2 mM sodium pyrophosphate, 2 mM sodium orthovanadate). The extent of phosphorylation of KDR is measured using an ELISA assay. The 96-well plates are coated with 1 μg per well of goat anti-rabbit antibody. Unbound antibody is washed off the plate and remaining sites are blocked with Superblock buffer (Pierce) prior to addition of the anti-flk-1 C-20 antibody (0.5 μg per plate, Santa Cruz). Any unbound antibody is washed off the plates prior to addition of the cell lysate. After a 2 hour incubation of the lysates with the flk-1 antibody, the KDR associated phosphotyrosine is quantitated by development with the HRP-conjugated PY-54 antibody and TMB, as described above. The ability of the compounds to inhibit the VEGF-stimulated autophosphorylation reaction by 50%, relative to VEGF-stimulated controls is reported as the IC₅₀ value for the test compound.

The ability of the compounds to inhibit mitogenesis in human endothelial cells is measured by their ability to inhibit ³H-thymidine incorporation into HUVE cells (human umbilical vein endothelial cells, Clonetics™). This assay has been well described in the literature (Waltenberger J et al. J. Biol. Chem. 269: 26988, 1994; Cao Y et al. J. Biol. Chem. 271: 3154, 1996). Briefly, 10⁴ cells are plated in collagen-coated 24-well plates and allowed to attach. Cells are re-fed in serum-free media, and 24 hours later are treated with various concentrations of compound (prepared in DMSO, final concentration of DMSO in the assay is 0.2% v/v), and 2-30 ng/ml VEGF₁₆₅. During the last 3 hours of the 24 hour compound treatment, the cells are pulsed with ³H thymidine (NEN, 1 μCi per well). The media are then removed, and the cells washed extensively with ice-cold Hank's balanced salt solution, and then 2 times with ice cold trichloroacetic acid (10% v/v). The cells are lysed by the addition of 0.2 ml of 0.1 N NaOH, and the lysates transferred into scintillation vials. The wells are then washed with 0.2 ml of 0.1 N HCl, and this wash is then transferred to the vials. The extent of ³H thymidine incorporation is measured by scintillation counting. The ability of the compounds to inhibit incorporation by 50%, relative to control (VEGF treatment with DMSO vehicle only) is reported as the IC₅₀ value for the test compound.

The activity of the compounds of formula 1, in vivo, can be determined by the amount of inhibition of tumor growth by a test compound relative to a control. The tumor growth inhibitory effects of various compounds are measured according to the methods of Corbett T. H., et al. “Tumor Induction Relationships in Development of Transplantable Cancers of the Colon in Mice for Chemotherapy Assays, with a Note on Carcinogen Structure”, Cancer Res., 35, 2434-2439 (1975) and Corbett, T. H., et al., “A Mouse Colon-tumor Model for Experimental Therapy”, Cancer Chemother. Rep. (Part 2)”, 5, 169-186 (1975), with slight modifications. Tumors are induced in the flank by s.c. injection of 1×10⁶ log phase cultured tumor cells suspended in 0.1-0.2 ml PBS. After sufficient time has elapsed for the tumors to become palpable (5-6 mm in diameter), the test animals (athymic mice) are treated with active compound (formulated by dissolution in appropriate diluent, for example water or 5% Gelucire™ 44/14 m PBS by the intraperitoneal (ip) or oral (po) routes of administration once or twice daily for 5-10 consecutive days. In order to determine an anti-tumor effect, the tumor is measured in millimeters with Vernier calipers across two diameters and the tumor volume (mm³) is calculated using the formula: Tumor weight=(length×[width]²)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). The flank site of tumor implantation provides reproducible dose/response effects for a variety of chemotherapeutic agents, and the method of measurement (tumor diameter) is a reliable method for assessing tumor growth rates.

Administration of the compounds of the present invention can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the compounds of the present invention administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

Advantageously, the present invention also provides kits for use by a consumer for treating disease. The kits comprise a) a pharmaceutical composition comprising a compound of the present invention and a pharmaceutically acceptable carrier, vehicle or diluent; and b) instructions describing a method of using the pharmaceutical composition for treating the specific disease.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc . . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds the patient when the next dose is to be taken.

In still another embodiment of the kits, the pharmaceutical composition may also comprise an additional compound that can be used in combination with a compound of the present invention, or the kit may comprise two pharmaceutical compositions: one containing a compound of the present invention and another containing an additional compound that can be used in combination with a compound of the present invention.

The compounds of the present invention may be applied as a sole therapy or may involve one or more other anti-tumor substances, for example those selected from, for example, mitotic inhibitors, for example vinblastine; alkylating agents, for example cisplatin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Administration of a combination of a compound of the present invention and an additional compound or additional compounds means that these compounds can be administered together as a composition or as part of the same unitary dosage form or in separate dosage forms, administered at the same time or at different times.

Exemplary parenteral administration forms include solutions or suspensions of the compounds of the present invention in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the compounds of the present invention therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of the compounds of the present invention are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, it was performed using a Waters Alliance HPLC system (2690+996 photodiode array). Preparative HPLC was performed using a Waters 717 autosampler, 996 PDA, 600 controller. Other details regarding the chromatographic procedures are provided within the examples below.

### EXAMPLE 1

Radiolabelled Compound

[¹⁴C] labeled monohydrochlroide salt of 3-(4-bromo-2,6-difluoro-benzyloxy)-5-[3-(4-pyrrolidin-1-yl-butyl)-ureido]-isothiazole-4-carboxylic acid amide (specific activity; 0.87 μCi/mg salt form) showed a radiochemical purity of >99%. The position of ¹⁴C-label is shown below.

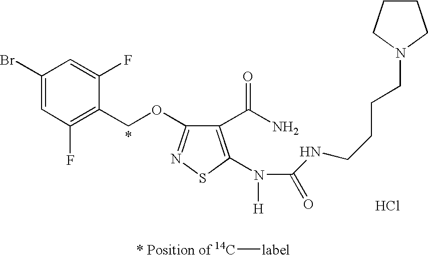

The labeled compound can be made in accordance with Scheme 1 set forth below.

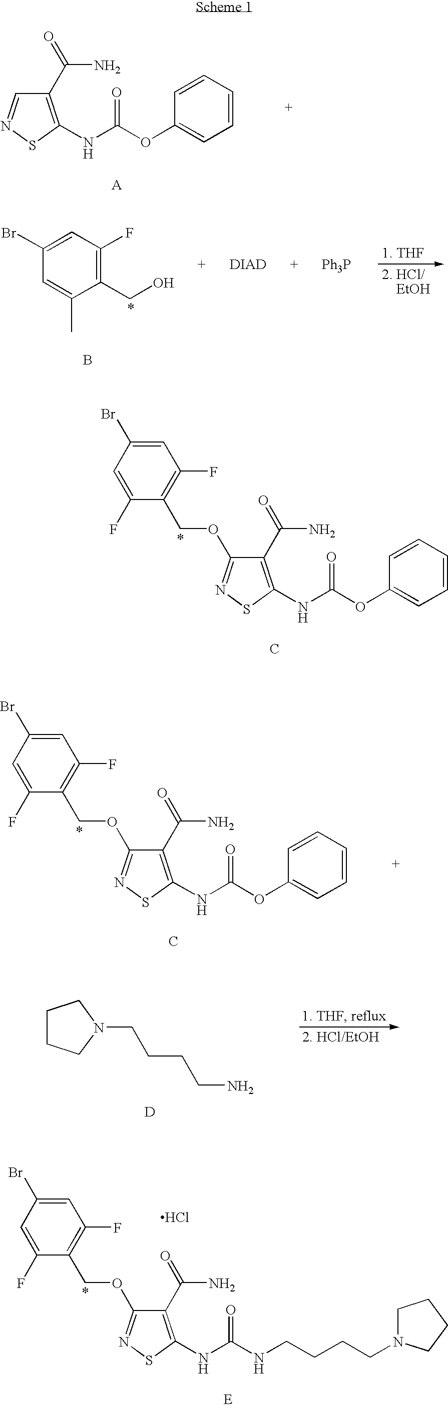

The synthesis of the labeled compound commenced with the Mitsunobu reaction of labeled compound B (43.6 mCi, 26.0 mCi/mmol) with compound A in the presence of triphenylphosphine and diisopropyl azodicarboxylate, as shown in the above scheme. When the reaction was complete, the solvent was displaced with acetonitrile and the salt formed by heating the resulting solid, i.e., the labeled free base form of compound C in an aqueous HCl/THF solution to yield the labeled hydrochloride salt (labeled compound C). Labeled compound C (12 mCi, 26.0 mCi/mmol) was then converted to the labeled free base form of compound E by transamidation with compound D. Salt formation to compound E (the hydrochloride salt) occurred following displacement of any reaction solvent with isopropyl alcohol. With salt formation a radiochemical purity of at least 99% was achieved.

Subjects and Dose Administration

Four normal healthy male subjects between the ages of 26 and 55 years participated in the study. The subjects entered the Clinical Research Facility 12 hours before dosing, and remained there for up to 384 hours after dosing under continuous medical observation. All subjects had fasted at least 8 hours and were given a single 100 mg oral dose of the labeled compound (˜93 μCi/subject). The dose was administered in an open fashion in the morning. A standard meal was provided 4 hours later. The dosing formulation was prepared by dissolving the radiolabeled material in citrate buffered vehicle that was prepared by adding 100 ml of water to a 4 oz. amber bottle containing a dry powder blend of citric acid and sodium citrate. Total volume of dosage given was 240 ml.

Sample Collection

After dosing, urine samples were collected for up to 16 days between the following time points: 0-12, 12-24, 24-48, 48-72, 72-96, 96-120, 120-144, 144-168, 168-192, 192-216, 216-240, 240-264, 264-288, 288-312, 312-336, 336-360 and 360-384 hours post-dose (i.e., first sample collected between 0-12 hours, second sample collected between 12-24 hours, and so on). The total volume of the urine samples was recorded after each collection. Feces were collected as passed, from time of dosing until 384 hours after dosing.

Blood sufficient to provided a minimum of 7 ml of plasma was collected, in heparinized tubes, from each subject at 0 (just before dosing), 1, 2, 4, 6, 8, 10, 12, 16, 24, 48, 72, 96, 120, 144 and 168 hours after the dose. Within 1 hour after collection, the blood samples were centrifuged in a refrigerated centrifuge and plasma was separated from whole blood. Samples collected over the first 168 hours post-dose were divided into two aliquots (3 ml and 4 ml). The 3 ml aliquots were used for the quantitation of unchanged 3-(4-bromo-2,6-difluoro-benzyloxy)-5-[3-(4-pyrrolidin-1-yl-butyl)-ureido]-isothiazole-4-carboxylic acid amide The other 4 ml aliquots were used for the analysis of total radioactivity. For metabolite identification, blood sufficient to yield 20 ml of plasma was collected at 4, 6, 8, 12, 16 and 24 hours after the dose. All samples were labeled, immediately frozen, and stored at −20° C. until analysis.

Determination of Radioactivity

Radioactivity in urine, feces, and plasma was measured by liquid scintillation counting. All samples were counted in a Wallac 1409 liquid scintillation counter with quench correction determined by monitoring the Compton edge of the external gamma source. Radioactivity less than twice the background value of the pre-dose matrix was considered to be below the limit of quantification.

Triplicate aliquots of plasma (0.5 ml) and urine (0.1 or 1.0 ml) for each sampling time were mixed with either 6 or 18 ml of Ecolite (+) scintillation cocktail and counted for radioactivity in a Wallac 1409 liquid scintillation counter.

Fecal samples for each sampling time were mixed with an equal weight of water in stomacher 400 bags and homogenized using a Stomacher homogenizer to obtain homogenous slurry. Triplicate aliquots of fecal homogenates (0.08 to 0.4 g) were air-dried overnight at room temperature prior to combustion with a Packard 307 oxidizer equipped with Oximate 80 robotics. During combustion, the liberated radioactive CO₂ was trapped in a column filled with 10 ml of a carbon dioxide absorbent (i.e. Carbo-Sorb E) forming a carbamate. This carbamate was flushed from the oxidizer with 8 ml of the liquid scintillation cocktail Permafluor E+. The amount of radioactivity in fecal homogenates was determined using a Wallac 1409 liquid scintillation counter and then corrected for combustion efficiency. Combustion efficiency of the Packard 307 oxidizer was monitored with commercially prepared Spec Check containing a known amount of carbon-14 radioactivity. Triplicate sets of 100 μL Spec Check were combusted at the beginning, mid point, and end of all oxidizer runs to ensure greater than 96% efficiency across each sample combustion session.

Radioactivity in the dose was expressed as 100% and the radioactivity in urine and feces at each sampling time was defined as the percentage of dose excreted in the respective matrices at that sampling time. The amount of radioactivity in plasma was expressed as nanogram-equivalents of parent drug per milliliter and was calculated by using the specific activity of the dose (0.93 μCi/mg base form) administered.

Extraction of Metabolites from Biological Samples

Urine samples obtained between the following time points were pooled for each subject: 0-12, 12-24, 24-48, 48-72 hours after the dose (i.e., first sample collected between 0-12 hours, second sample collected between 12-24 hours, and so on). The pooled samples (˜100 ml) were lyophilized overnight. The residues were extracted with methanol twice (10 and 5 ml). The supernatants were combined and 0.2 ml aliquots were counted in a liquid scintillation counter to determine the extraction efficiency. The supernatants were evaporated to dryness in a Turbo Vap LV evaporator. The residues were reconstituted with 1 ml of MeOH. Aliquots (50 μl) were injected onto the HPLC column without further purification.

Fecal homogenates were pooled (up to 0-264 hours) for each subject. The pooled samples (˜10 g) were suspended in 25 ml of acetonitrile. The suspensions were sonicated for 10 min, then stirred on a magnetic stirrer for 1 hour and centrifuged. Supernatants were separated and the residues further extracted with 25 ml of acetonitrile. The two supernatants were combined and 1 ml aliquots were counted in a scintillation counter to determine the extraction efficiency. The organic solvents were evaporated to dryness in a Turbo Vap LV evaporator and the residues were reconstituted in 1 ml of MeOH:water (50:50). Aliquots (100 μl) of concentrated fecal extracts were injected onto the HPLC column.

Plasma samples were pooled at 4, 6, 8, 12, 16 and 24 hours post-dose according to the published method (Hamilton et. al., 1981) and the pooled plasma samples (15 ml) were mixed with 35 ml of acetonitrile, vortexed and sonicated. The mixtures were centrifuged and the supernatants removed. The pellets were extracted with 10 ml of acetonitrile. The supernatants were combined and aliquots (1 ml) were counted by a liquid scintillation counter to determine the extraction efficiency. The organic solvents were evaporated to dryness in a nitrogen Turbo Vap LV evaporator and the residues were reconstituted in 300 □l acetonitrile:water (20:80). Aliquots (100 μl) were injected onto the HPLC column connected to LC-accurate radioisotope counting (ARC; AIM Research Co. Hockessin, Del.) system.

Quantitative Assessment of Metabolites

Quantitation of the urinary and circulating metabolites was carried using LC-ARC system using a 2.2 ml liquid flow cell. Quantitation of the fecal metabolites was carried out by measuring the radioactivity in the individual peaks that were separated on HPLC using β-Ram (IN/US, Win-flow). The β-Ram provided an integrated printout in CPM and the percentage of the radiolabeled material, as well as peak representation. The β-Ram was operated in the homogeneous liquid scintillation counting mode with the addition of 3 ml/min of Tru-Count (IN/US) scintillation cocktail to the effluent post-UV-detection.

High Performance Liquid Chromatography

The HPLC system consisted of a HP-1050 solvent delivery system, a HP-1050 membrane degasser, an HP-1050 autoinjector (Hewlett Packard), a Thermo Separations spectromonitor 3200 UV and an IN/US radioactive monitor (β-RAM). Chromatography was performed on a Kromasil C-18 column (4.6 mm×150 mm, 5 μm). The mobile phase was composed of 5 mM ammonium formate (pH=3.0) (solvent A) and acetonitrile (solvent B). The solvent delivery gradient program was as follows:

| Time (min) | Solvent A | Solvent B |
|---|---|---|
| 0 | 95 | 5 |
| 5.0 | 95 | 5 |
| 5.2 | 75 | 25 |
| 40 | 60 | 40 |
| 50 | 20 | 80 |
| 52 | 95 | 5 |
| 60 | 95 | 5 |

The system was allowed to equilibrate for 8 min prior to the next injection. A flow rate of 1 ml/min was maintained throughout the analysis.

Mass Spectrometry

Identification of the metabolites was performed on a Finnigan LCQ Deca LC/MS/MS operating with electrospray (ESI). The effluent from the HPLC column was split and approximately 50 μl/min was introduced into the API interface. The remaining effluent was directed to the flow cell of β-RAM. The β-RAM response was recorded in real time by the mass spectrometer that provided simultaneous detection of radioactivity (RAD) and mass spectrometry data. The interface was operated at ±4500 V and the mass spectrometer was operated either in the positive mode.

Excreted Metabolites

An HPLC-radiochromatogram of human urinary metabolites indicated the presence of the starting 3-(4-Bromo-2,6-difluoro-benzyloxy)-5-[3-(4-pyrrolidin-1-yl-butyl)-ureido]-isothiazole-4-carboxylic acid amide and a total of 4 metabolites (M1, M2, M4 and M5). An HPLC-radiochromatogram of human fecal metabolites indicated the presence of starting 3-(4-Bromo-2,6-difluoro-benzyloxy)-5-[3-(4-pyrrolidin-1-yl-butyl)-ureido]-isothiazole-4-carboxylic acid amide and a total of 2 metabolites, M1 and M9.

An HPLC-radiochromatogram of circulating metabolites indicated the presence of the starting material 3-(4-bromo-2,6-difluoro-benzyloxy)-5-[3-(4-pyrrolidin-1-yl-butyl)-ureido]-isothiazole-4-carboxylic acid amide and a total of 2 metabolites, M4 and M9.

The metabolites M8 was detected in feces, bile and plasma of beagle dogs and Sprague-Dawley Rats using methods of isolation and detection similar to those used for human samples set forth above.

Identification of Metabolites

3-(4-Bromo-2,6-difluoro-benzyloxy)-5-[3-(4-pyrrolidin-1-yl-butyl)-ureido]-isothiazole-4-carboxylic acid amide (Parent drug): The parent drug had a retention time of 29.5-30.5 min on HPLC and showed a protonated molecular ion (M+H)⁺ at m/z 532. Its MS² and MS³ spectra showed fragment ions at m/z 126, 143, 169, 205, 364, 444 and 515. The ion at m/z 515 resulted from the loss of NH₃. The diagnostic ions at m/z 364 and 169 resulted from the cleavages of the pyrrolidinyl-butyl ureido bond with the charge retention on both sides. The ion at m/z 444 resulted from the losses of NH₃ and the pyrrolidinyl moiety. The ion at m/z 143 was from the pyrrolidinyl-butyl amino moiety. The ion at m/z 126 was due to the pyrrolidinyl-butyl moiety.

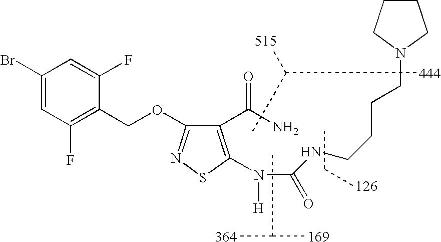

Metabolite M1: M1 was detected in urine and feces of humans. It had a retention time of 27.5-28.3 min on HPLC and showed a protonated molecular ion at m/z 564. Its MS/MS ion spectrum showed fragments at m/z 175, 201, 364, and 547. The ion at m/z 364, similar to that of the parent compound, and an ion at m/z 201, 32 mass units higher than the ion at m/z 169 of the parent compound, suggested that the modification had occurred on the pyrrolidinyl-butyl moiety. The ion at m/z 444, similar to that of the parent compound, further suggested that the oxidation had occurred on the pyrrolidinyl moiety. Treatment of M1 with diazomethane or MeOH/sulfuric acid resulted in the disappearance of M1 and appearance of a peak at a retention time of −34.0 min on HPLC. The new peak showed a protonated molecular ion at m/z 578, 14 mass units higher than M1, suggesting the presence of a carboxylic acid. Based on these data, the M1 was identified as 4-(4-{3-[3-(4-bromo-2,6-difluoro-benzyloxy)-4-carbamoyl-isothiazol-5-yl]-ureido}-butylamino)-butyric acid.

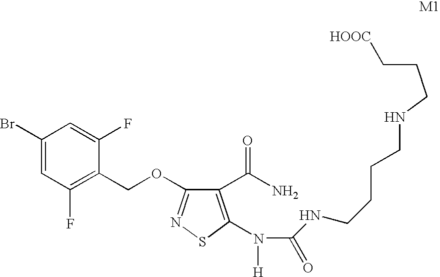

Metabolite M2: M2 was detected in urine of humans and in urine, feces, bile and plasma of Beagle dogs. It had a retention time of ˜31.5 min on HPLC and showed a protonated molecular ion at m/z 548, 16 mass units higher than the parent compound, suggesting that a single oxidation had occurred. Its MS² spectrum showed fragment ions at m/z 159, 185, 461, 513 and 531. The ion at m/z 531 resulted from the loss of NH₃. The ion at m/z 185 suggested that the single oxidation had occurred on the pyrrolidinyl-butyl moiety. The treatment of isolated M2 fraction from urine sample with titanium (III) chloride regenerated the parent compound. Based on these data, M2 was identified as 3-(4-bromo-2,6-difluoro-benzyloxy)-5-[3-(4-pyrrolidin-1-yl-N-oxide-butyl)-ureido]-isothiazole-4-carboxylic acid amide.

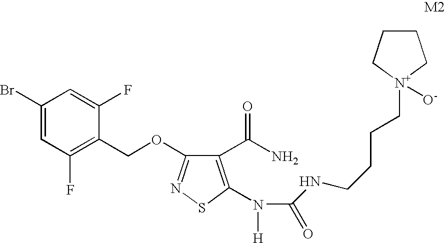

Metabolite M4: M4 was detected in urine and plasma. It had a retention time of 9.5-10.8 min on HPLC. Mass spectral data for M4 could not be obtained due to low abundance of this metabolite. However, its HPLC retention time was similar to that of metabolite M4 found in rat urine, and a synthetic standard (specially ordered from Aldrich; Catalog #L-44656-4; CAS#183065-68-1). Based on these data, M4 was identified as 4-bromo-2,6-difluorobenzoic acid.

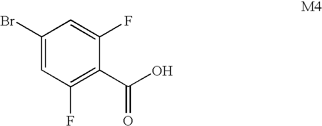

Metabolite M5: M5 was detected in urine of humans and beagle dogs. It had a retention time of −22.8 min on HPLC and showed a deprotonated molecular ion at m/z 366, lower than the parent compound, suggesting that M5 was a cleaved product. Its MS/MS spectrum showed fragment ions at m/z 217, 237 and 319. The ion at m/z 237 was from the bromo-difluorobenzylthiol moiety. Treatment of M5 from urine with diazomethane resulted in the disappearance of M5 and appearance of a peak at a retention time of −35 min on HPLC. The new peak showed a protonated molecular ion at m/z 382, 14 mass units higher than M5, suggesting the presence of a carboxylic acid. Its MS/MS spectrum showed fragment ions at m/z 144, 280, 309, 322, 340 and 350. M5 had identical spectrum and had similar HPLC retention time to that of the synthetic standard, which was prepared by Scheme 2 shown below.

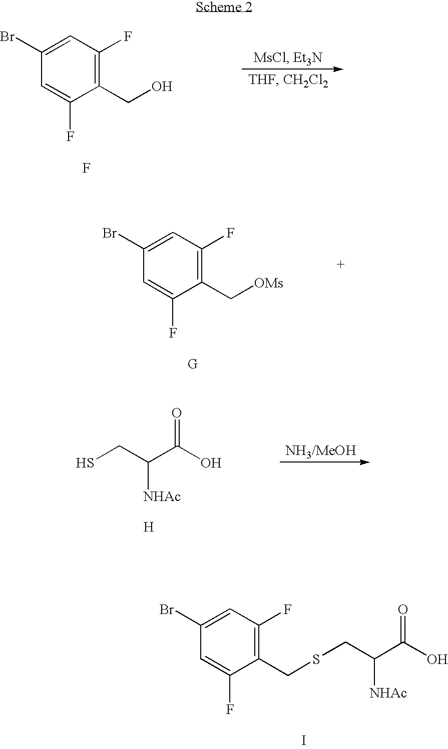

Formation of the mesylate of benzyl alcohol F under standard conditions, followed by displacement of the mesylate with thiol H under mildly basic conditions resulted in the formation of compound 1, which was identical to M5 under all analytical conditions evaluated. Based on these data, M5 was identified as an N-acetylcysteine conjugate of bromo-difluorobenzyl alcohol, i.e., 2-Acetylamino-3-(4-bromo-2,6-difluoro-benzylsulfanyl)-propionic acid

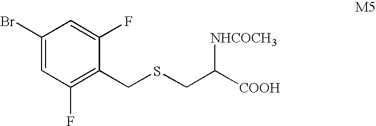

Metabolite M8: M8 was detected in feces, bile and plasma of beagle dogs. It has a retention time of 30.4-38.2 min on HPLC and showed a protonated molecular ion at m/z 544. Its MS/MS spectrum showed fragment ions at m/z 155, 166, 181, 205, 364, 501 and 527. The ion at m/z 527 resulted from the loss of NH₃. The ion at m/z 364, similar to that of parent compound, suggested that the bromo-difluorobenyloxy-isothiazole-carboxylic acid amide moiety was unchanged. The ion at m/z 155, 12 Daltons higher than that of the parent compound, suggested that the modification has occurred on the pyrrolidinyl-butyl moiety. Based on these date, M8 was tentatively identified as 3-(4-bromo-2,6-difluoro-benzyloxy)-5-{3-[4-(2-oxo-2,5-dihyro-pyrrol-1-yl)-butyl]-ureido}-isothiazole-4-carboxylic acid amide, having the structure shown below.

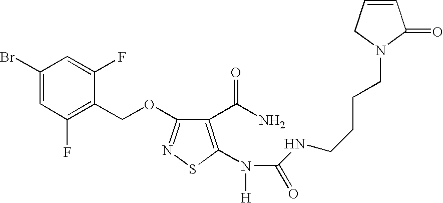

Metabolite M9: M9 was detected in feces and plasma. It had a retention time of −45.1 min. Mass spectral data for M9 could not be obtained due to low abundance of this metabolite. However, its HPLC retention time was similar to that of metabolite M9 found in dog feces. Based on these data, M9 was identified as 4-{3-[3-(4-bromo-2,6-difluorobenzyloxy)-4-carbamoyl-isothiazol-5-yl]-ureido}-butyric acid.

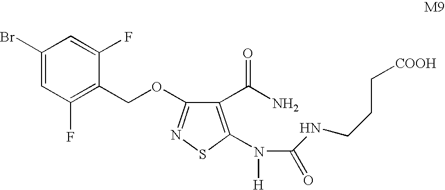

In addition to being isolated as metabolites of 3-(4-bromo-2,6-difluoro-benzyloxy)-5-[3-(4-pyrrolidin-1-yl-butyl)-ureido]-isothiazole-4-carboxylic acid amide, the compounds of the present invention (except for 2-Acetylamino-3-(4-bromo-2,6-difluoro-benzylsulfanyl)-propionic acid (M5), which may be prepared as set forth above in Scheme 2) may be synthetically prepared according to the general procedures disclosed in International Publication WO 99/62890, published Dec. 9, 1999, and using an appropriate amine to arrive the desired final structure. More specifically Scheme 1, as set forth above may be followed except that unlabeled compounds may be used and a suitable amine in place of the amine D may be used to arrive at the desired compound. In some cases, use of these amines will require the presence of protecting groups, well known to those skilled in the art. In these cases, suitable protection/deprotection steps will be added to the procedure outlined in Scheme 1.

1. A substantially pure compound of the formula 1 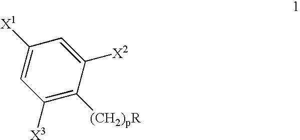
wherein:
X¹, X² and X³ independently is a halogen; p is an integer from 1 to 3; and R is OR¹ wherein R¹ is a substituted isothiazole ring or SR² wherein R² is (CH₂)_{q}C(COOH)(NHC(O)R³) wherein q is an integer from 0 to 3, and R³ is (C₁-C₄)alkyl; or a pharmaceutically acceptable salt, solvate or prodrug thereof.

2. The compound according to claim 1, wherein said compound is represented by the compound of formula 2 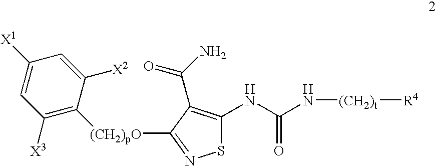
wherein t is an integer from 3 to 5; and R⁴ is —COOH, —NH(CH₂)ₖOH wherein k is an integer from 1 to 5, —NH(CH₂)ₙCOOH wherein l is an integer from 1 to 5, or a 5-membered nitrogen containing heterocycle, which may optionally be substituted by one or more oxo (═O) moieties, hydroxyl groups, or wherein an oxygen atom is bonded to the nitrogen atom of the 5-membered nitrogen containing heterocycle to form an N-oxide (N—O) group.

3. The compound according to claim 1, wherein X¹ is bromine, X² and X³ are fluorine and p is 1.

4. The compound according to claim 3, wherein t is 4.

5. The compound according to claim 4, wherein R⁴ is a 5-membered nitrogen containing heterocycle.

6. The compound according to claim 5, wherein R⁴ is represented by the formula

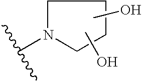

7. The compound according to claim 5, wherein R⁴ is represented by the formula

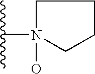

8. The compound according to claim 5, wherein R⁴ is represented by the formula

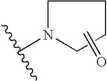

9. The compound according to claim 5, wherein R⁴ is represented by the formula

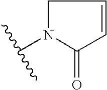

10. The compound according to claim 1 selected from the group consisting of
4-(4-{3-[3-(4-bromo-2,6-difluoro-benzyloxy)-4-carbamoyl-isothiazol-5-yl]-ureido}-butylamino)-butyric acid;
3-(4-bromo-2,6-difluoro-benzyloxy)-5-[3-(4-pyrrolidin-1-yl-N-oxide-butyl)-ureido]-isothiazole-4-carboxylic acid amide;
2-Acetylamino-3-(4-bromo-2,6-difluoro-benzylsulfanyl)-propionic acid;
2-Acetylamino 3-(4-bromo-2,6-difluoro-benzylsulfanyl)-2-methyl-propionic acid;
3-(4-Bromo-2,6-difluoro-benzyloxy)-5-{3-[4-(4-hydroxy-butylamino)-butyl]-ureido}-isothiazole-4-carboxylic acid amide;
4-{3-[3-(4-bromo-2,6-difluorobenzyloxy)-4-carbamoyl-isothiazol-5-yl]-ureido}-butyric acid;
3-(4-Bromo-2,6-difluoro-benzyloxy)-5-{3-[4-(2,3-dihydroxy-pyrrolidin-1-yl)-butyl]-ureido}-isothiazole-4-carboxylic acid amide;
3-(4-Bromo-2,6-difluoro-benzyloxy)-5-{3-[4-(2,4-dihydroxy-pyrrolidin-1-yl)-butyl]-ureido}-isothiazole-4-carboxylic acid amide;
3-(4-Bromo-2,6-difluoro-benzyloxy)-5-{3-[4-(3,4-dihydroxy-pyrrolidin-1-yl)-butyl]-ureido}-isothiazole-4-carboxylic acid amide;
3-(4-Bromo-2,6-difluoro-benzyloxy)-5-{3-[4-(2,5-dihydroxy-pyrrolidin-1-yl)-butyl]-ureido}-isothiazole-4-carboxylic acid amide;
3-(4-bromo-2,6-difluoro-benzyloxy)-5-{3-[4-(2-oxo-2,5-dihydro-pyrrol-1-yl)-butyl]-ureido}-isothizole-4-carboxylic acid amide;
and the pharmaceutically acceptable salts, prodrugs, hydrates and solvates of the foregoing compounds.

11. A pharmaceutical composition for the treatment of a hyperproliferative disorder in a mammal which comprises a therapeutically effective amount of a compound according to claim 1 and a pharmaceutically acceptable carrier.

12. The pharmaceutical composition of claim 11, wherein said disorder is a non-cancerous hyperproliferative disorder.

13. The pharmaceutical composition of claim 12, wherein said disorder is a benign hyperplasia of the skin or prostate.

14. A method of treating a hyperproliferative disorder in a mammal which comprises administering to said mammal a therapeutically effective amount of a compound according to claim 1.

15. A method for the treatment of a hyperproliferative disorder in a mammal which comprises administering to said mammal a therapeutically effective amount of a compound according to claim 1 in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, NK1 receptor antagonist, 5-HT₃ receptor antagonist, COX-2 inhibitor, an EGFR inhibitor, and anti-androgens.
